(19) United States
(12) Patent Application Publication
(10) Pub. No.: US 2023/0048417 A1
(43) Pub. Date: Feb. 16, 2023

(54) DENTAL IMPLANT

(71) Applicant: SUDIMPLANT, Toulouse (FR)

(72) Inventor: Olivier BENHAMOU, Brussels (BE)

(73) Assignee: SUDIMPLANT, Toulouse (FR)

(21) Appl. No.: 17/883,845

(22) Filed: Aug. 9, 2022

Related U.S. Application Data
(63) Continuation of application No. 16/219,003, filed on Dec. 13, 2018, now abandoned.

(30) Foreign Application Priority Data
Dec. 13, 2017 (BE) 2017/0171

(51) Int. Cl. A61C 8/00

(52) U.S. Cl. CPC A61C 8/0013; A61C 8/0012; A61C 8/0037; A61C 8/0083; A61C 8/0022; A61C 8/0093

(57) ABSTRACT

A dental implant has a core manufactured in metal and a sleeve having an internal wall and nested by sliding on an upper part of the core in such a manner that the internal wall is located faced to that upper part of the core, which sleeve is manufactured from ceramic or Peek, a gap being left between the core and the internal wall of the sleeve in such a manner that an adhesive layer can be lodged in a space formed by the presence of that gap, wherein said gap has a distance situated between 5 and 30 μm, in particular between 10 and 20 μm.

The present invention relates to a dental implant comprising a core manufactured in metal and a sleeve having an internal wall and nested by sliding on an upper part of the core in such a manner that the internal wall is located faced to that upper part of the core, which sleeve is manufactured from ceramic or Peek, a gap being left between the core and the internal wall of the sleeve in such a manner that an adhesive layer can be lodged in a space formed by the presence of that gap.

Such a dental implant is known from the patent EP 1 418 858. In the known dental implant the sleeve is glued on the core. To this purpose a gap situated between 80 and 150 μm is left between the internal wall of the sleeve and the upper part of the core. That gap enables to form a sufficient space for enabling a good elasticity between the sleeve and the core.

A drawback of the known implant is that there is a risk to unstick. Indeed, it has been established that when or after the insertion of these implants in the dense or irregular jawbone, the latter can sometimes push the sleeve back and thus cause that the adhesive looses, thereby causing that the sleeve detaches from the core. Those implants were therefore not suitable for being completely lodged in the jawbone, and the sleeve could not be in contact with the bone.

The object of the invention is to realise a dental implant where the risk of unsticking and breaking are considerably reduced.

To this purpose an implant according to the invention is characterised in that said gap has a distance situated between 5 and 30 μm, in particular between 10 and 20 μm. This value of the gap enables in an unexpected manner to combine on the one hand a good resistance at the insertion and at the contact with the bone, and on the other hand to keep sufficient space between the core and the sleeve for enabling a certain elasticity between the sleeve and the core. This value of the gap also enables a better result at a pulling test of the sleeve fixed on the core and reduces the probability of a break at this test. That sleeve can then be partially or integrally embedded in the bone, and that implant can also be totally embedded in the jawbone, even when irregular.

### BRIEF DESCRIPTION OF THE DRAWING

FIG. 1 is a cross sectional view of a dental implant according to the invention.

A first preferred embodiment of a dental implant according to the invention is characterised in that the ceramic of which the sleeve is manufactured is Zirconia. Zirconia is a material which has excellent bio-compatible properties, in particular when in contact with soft tissues.

A second preferred embodiment of a dental implant according to the invention is characterised in that the adhesive layer is a biocompatible bi-component epoxy adhesive layer. This adhesive is well appropriated for dental implant use.

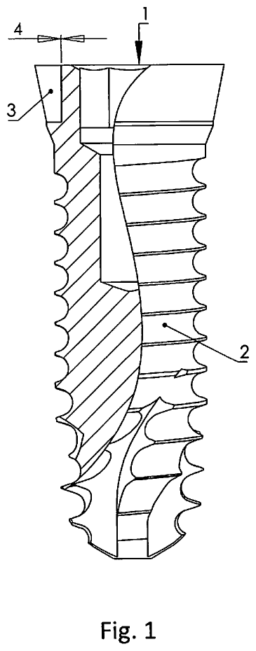

The invention will now be described in more details with respect to the sole attached FIG. 1 and which shows an embodiment of a dental implant according to the invention.

FIG. 1 illustrates a cross section of a dental implant 1 according to the invention. The dental implant comprises a core 2 manufactured from metal, in particular titanium. Titanium has the advantage of being resistant, flexible and bio-compatible for use as a dental implant in a patient's mouth. The core has preferably an external screw thread enabling to screw the core in the jawbone.

The sleeve 3 is placed on an upper part of the core and extends over the whole circumference of the core. The sleeve has an internal wall and is nested by sliding on an upper part of the core, in such a manner that the internal wall is located faced to that upper part of the core when the implant is implanted in the jawbones of the patient, the sleeve is positioned in the gingival between the bone and the mouth cavity.

The sleeve is preferably manufactured in ceramic, more particularly in Zirconia. The sleeve can also be manufactured in Peek.

A gap 4 is left between the core and the internal wall of the sleeve in such a manner that an adhesive layer can be lodged in a space formed by the presence of that gap. The gap has a distance situated between 5 and 30 μm, in particular between 10 and 20 μm. That gap extends over the whole height of the sleeve in such a manner that on the one hand the nesting by sliding of the sleeve on the upper part of the core is facilitated, and on the other hand for enabling an adhesive layer to extend over this whole height and thus enabling a good adherence and good assembling between the sleeve and the core.

The used adhesive for adhering the sleeve at the core is preferably a bi-component epoxy adhesive. This adhesive is well accepted for use in a mouth of dental implants, as it is bio-compatible.

The advantage of leaving a gap having a distance situated between 5 and 30 μm, in particular between 10 and 20 μm, is that it enables in an unexpected manner to combine on the one hand a good resistance of that sleeve when in contact with the jawbone, and on the other hand to keep sufficient space between the core and the sleeve for enabling a certain elasticity between the sleeve and the core for reasons of the applied chewing forces. Indeed, a problem of detachment of the sleeve from the core has been established on dental implants known from the prior art. An in depth analysis of the problem has made it possible to establish that the jawbone can sometimes push the sleeve of the implant back when implanted in the patient's mouth. This is namely due to the fact that the bone is practically never perfectly flat and that even some small irregularities in the bone crest, in the order of less than one millimetre, already causes a pression on the sleeve. Thus, this pression applied by the jawbone on the sleeve can cause that the adherence and the assembling loose when applying the implant, thereby causing that the sleeve leaves the core.

This value of the gap having a distance situated between 5 and 30 μm, in particular between 10 and 20 μm, enables also a better result to the pulling test of the sleeve fixed on the core and reduces the probability of breaking at this test. Those tests are realised at the manufacturing plant at manufacturing of the implant in order to verify the pulling resistance of the sleeve fixed at the core. Those tests enabled to establish that even with a thin adhesive layer a sufficient elasticity is obtained between the sleeve + and the core for avoiding a breaking of the sleeve.

Another advantage of that gap according to the invention is that the risk of bacterial infiltration in the space left by that gap is minimized. Indeed, with respect to this very small distance of the gap and due to the fact that the space formed by that gap is filled with that bio-compatible adhesive, there remains as if to say no more place for bacterial.

1. A dental implant comprising a core manufactured in metal and a sleeve having an internal wall and nested by sliding on an upper part of the core in such a manner that the internal wall is located faced to that upper part of the core, which sleeve is manufactured from ceramic or Peek, a gap being left between the core and the internal wall of the sleeve, characterised in that said gap has a distance situated between 10 and 20 μm and an adhesive is lodged within the gap for adhering the sleeve to the core and together with the gap distance enabling elasticity between the sleeve and the core.

2. The dental implant as claimed in claim 1, characterised in that the ceramic of which the sleeve is manufactured is Zirconia.

3. The dental implant as claimed in claim 1, characterised in that the adhesive is a biocompatible bi-component epoxy adhesive.

4. The dental implant as claimed in claim 3, characterised in that the ceramic of which the sleeve is manufactured is Zirconia.
